(19) United States
(12) Patent Application Publication
(10) Pub. No.: US 2020/0164129 A1
(43) Pub. Date: May 28, 2020

(54) DIALYSIS DEVICE COMPRISING AN EVAPORATOR/CONDENSER DEVICE

(71) Applicant: TRIOMED AB, Lund (SE)

(72) Inventor: Anders WALLENÅS, Lomma (SE)

(73) Assignee: TRIOMED AB, Lund (SE)

(21) Appl. No.: 16/630,751

(22) PCT Filed: Jul. 13, 2018
(86) PCT No.: PCT/SE2018/050765

§ 371 Date: Jan. 13, 2020

(30) Foreign Application Priority Data
Jul. 14, 2017 (SE) 1730196-1

(51) Int. Cl. A61M 1/16; A61M 1/28; A61M 1/34; B01D 5/00

(52) U.S. Cl. CPC A61M 1/1696; A61M 1/1656; B01D 5/006; A61M 1/3413; A61M 1/3472; A61M 1/287

(57) ABSTRACT

A dialysis device comprising a dialysis circuit for providing dialysis fluid and returning purified fluid to the dialysis circuit. The device comprises an evaporator/condenser device, provided with an evaporator bag and a condenser bag. The evaporator bag receives dialysis fluid from the dialysis bag and produces steam, whereby a concentrated dialysis fluid is left and drained to a drain bag. The condenser bag receives the steam produced in the evaporation bag and condenses the steam for producing pure water for being returned to said dialysis circuit. The evaporation and condensation takes place at a subatmospheric pressure, such as between 30 and 70 mmHg at a temperature of about 30 to 44° C. Heat energy is provided to the evaporation bag and the condenser bag is cooled. The cooled energy may be absorbed by a heat pump for delivery of heat to the evaporator bag.

### FIELD OF INVENTION

The present invention relates to a method and device for performing dialysis. The device and method are specifically advantageous at peritoneal dialysis but may also be used at hemodialysis and hemofiltration.

### BACKGROUND

Traditional peritoneal dialysis is performed as Continuous Ambulatory Peritoneal Dialysis, CAPD, wherein a fresh peritoneal dialysis solution is installed or filled in the peritoneal cavity and is allowed to dwell in the peritoneal cavity for a certain time period, for example 4 hours. Thereafter, the spent dialysis solution is drained and new fresh dialysis solution is filled into the peritoneal cavity.

Peritoneal dialysis is particularly suitable for patients still having partially functioning kidneys, for example since any residual kidney function may be maintained, compared to hemodialysis. However, CAPD normally has the disadvantage of relatively poor urea removal and low ultrafiltration.

Another peritoneal dialysis modality is APD, automated peritoneal dialysis, wherein a cycler is arranged for filling and draining the peritoneal cavity, for example during night time. In order to obtain sufficient urea or toxin removal, large quantities of fluid is required, such as 15 liter or more per day.

Hemodialysis is performed by extracting blood from a patient into an extracorporeal circuit comprising a dialyzer and a dialysis machine. Blood is passed into a compartment of the dialyzer and a dialyzer fluid is passed into another compartment of the dialyzer, whereby the compartments are separated by a semipermeable membrane. Large quantities of fluid is required, such as 40 liter or more per day.

Such large quantities of dialysis fluid may be produced by providing ultrapure water and mixing said ultrapure water with a sterile concentrate in a dialysis machine, such as A-concentrate and B-concentrate. The ultrapure water is provided by a water unit, which may comprise a reverse osmosis membrane for removal of ions, molecules and toxins from the water. The water unit often supply ultrapure water to several dialysis machines in a dialysis center.

Another less conventional methods of producing ultrapure water is distillation followed by condensation. A further unconventional method of producing ultrapure water is electrolysis for forming oxygen and hydrogen, which are recombined for forming ultrapure water. Patent publication WO2016132187A1 describes a hemodialysis system using both the last-mentioned methods for producing fresh water having a high degree of purity from water which drains out of the dialysis machine.

In order to make a peritoneal dialysis device wearable, constructions in which the dialysis fluid is regenerated has been provided. A filter may be arranged for adsorption of toxic products, such as urea and creatinine, whereupon the regenerated fluid is filled back to the peritoneal cavity. Filter dialysis has also been suggested for hemodialysis.

Peritoneal fluids normally comprise an ultrafiltration capacity, provided by a substance such as glucose or other large molecule, generating an osmotic or oncotic pressure gradient. Ultrafiltration fluid is removed from the peritoneal cavity into a drain bag. However, the concentration of for example urea in the drain bag is much lower compared to for example normal urine.

### SUMMARY OF THE INVENTION

Accordingly, an object of the present invention is to mitigate, alleviate or eliminate one or more of the above-identified deficiencies and disadvantages singly or in any combination.

In a first aspect, there is provided a dialysis device comprising a dialysis circuit for providing dialysis fluid and returning purified fluid to the dialysis circuit, and an vaporator/condenser device (30), which comprises: an evaporator bag for receiving dialysis fluid from the dialysis circuit and for evaporation thereof for producing steam; and a condenser bag for receiving said steam produced in the evaporation bag and condensing the steam for producing pure water in said condenser bag for returning to said dialysis circuit; further comprising a drain bag for receiving a concentrated ultrafiltration fluid produced in the evaporator bag. The evaporation and condensation may take place at a subatmospheric pressure, such as between 30 and 70 mmHg.

The produced pure water may be conditioned by a concentrate fluid before being returned to the dialysis circuit, wherein said concentrate fluid comprises at least one of: sodium ions, potassium ions, calcium ions, magnesium ions, chloride ions, bicarbonate ions, acetate, glucose.

The dialysis device may be operated batchwise, so that a predetermined amount of dialysis fluid is evaporated and condensed in a single step.

The dialysis process may be hemofiltration dialysis, wherein the circuit may comprise a hemofilter providing said dialysis fluid and wherein said pure water, conditioned with concentrate fluid, is provided as postdilution or predilution.

The dialysis process may be peritoneal dialysis.

The ultrafiltration fluid, which is drained to a drain bag, may be concentrated at least five times, such as eight times or ten times.

The evaporation and condensation may take place at substantially the same pressure.

### BRIEF DESCRIPTION OF THE DRAWINGS

Further objects, features and advantages of the invention will become apparent from the following detailed description of embodiments of the invention with reference to the drawings, in which:

FIG. 1 is a schematic view of a first embodiment of the invention.

FIG. 2 is a schematic view of a second embodiment of the invention.

FIG. 3 is a schematic view of a third embodiment of the invention.

FIG. 4 is a schematic view of a fourth embodiment of the invention.

FIG. 5 is a schematic view of a fifth embodiment of the invention.

FIG. 6 is a schematic view of a sixth embodiment of the invention.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

Embodiments of the invention are based on an evaporation/condenser device which is operated at a low pressure for producing pure water and an ultrafiltration dialysis fluid, which is drained to a drain bag and eventually discarded. The evaporation/condenser device performs a dual purpose, namely to concentrate the ultrafiltration fluid to be drained and to generate a “sterile” fluid to be used for the dialysis process, either hemodialysis, hemofiltration, hemodiafiltration or peritoneal dialysis and any combinations thereof.

By concentration of the ultrafiltration fluid to be drained and discarded, large amount of waste products may be collected therein, in particular urea and creatinine but also middle molecule substances such as beta-2-microglobulin and endotoxins. In addition, plasma ions are concentrated in the ultrafiltration fluid, and these ions may be required to be replaced by a concentrate fluid to be added to the pure water produced. Such plasma ions are sodium ions, potassium ions, calcium ions, magnesium ions, chloride ions, bicarbonate ions, acetate, glucose, etc.

The pure water produced by the evaporation/condenser device may be less than 100% pure, since water droplets may pass through the system. However, bacteria will not pass the system, which means that the produced pure water is sufficiently pure or sterile for infusion into the blood. Depending on the process, the pure water is 95% pure water or more than 99% pure water.

Since the discarded ultrafiltration fluid is concentrated, no further purification of the dialysis fluid is normally required. In particular, the present embodiments comprising the evaporation/condenser device may fully (or partly) replace adsorption filters or cartridges used during adsorption dialysis, which are popular in wearable peritoneal dialysis products.

The evaporation/condenser device requires large amounts of heat energy, normally in the form of electric energy. Such large amounts of energy are not easily provided by batteries. However, the rest of the device has a relatively low weight and may be wearable. Thus, the device may be adapted and arranged in the morning and carried all the day. When the person is positioned adjacent a line outlet, such as during work hours, the device may be connected and perform dialysis. Since such power outlets are located almost everywhere, dialysis may be performed for almost the entire day. During night-time, the device may be connected all the time.

Embodiments of the invention will be described below with reference to the drawings. Several features are described in different embodiments, but each and every feature may be included in any of the other embodiments.

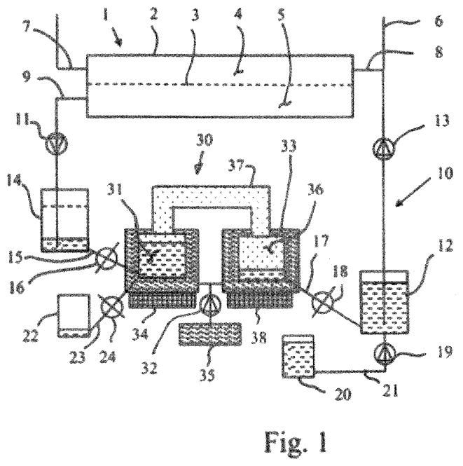

FIG. 1 discloses a first embodiment of the invention. The device 10 is connected to a hemofilter 1 of conventional design, which is intended to be connected to a patient for performing hemofiltration. The hemofilter may be a conventional hollow fiber filter or a plate filter.

The hemofilter 1 comprises an enclosure 2, comprising a semipermeable membrane 3 dividing the enclosure into a first compartment 4 and a second compartment 5. The first compartment 4 is included in an extracorporeal blood circuit 6, in which blood from a patient is circulated in a conventional manner during dialysis. Blood enters the first compartment 4 via a blood inlet 7 and exits the first compartment 4 via a blood outlet 8. A filtrate exits the second compartment 5 via a filtrate outlet 9.

The filtrate is removed from the blood circuit via the semipermeable membrane and the filtrate outlet 9 by means of a filtrate pump 11, which controls the amount of fluid filtrated.

A sterile fluid in a fluid bag 12 is infused in the extracorporeal circuit adjacent the blood outlet 8 by means of an infusion pump 13, resulting in so called postinfusion of fluid. The infusion pump 13 may alternatively infuse the sterile fluid in the extracorporeal circuit adjacent the blood inlet 7, resulting in so called preinfusion of fluid (not shown).

The infusion of fluid via infusion pump 13 is controlled so that a desired dialysis dose is provided. The removal of fluid by filtrate pump 11 from the extracorporeal circuit is controlled so that a desired ultrafiltration is obtained.

Below, the operation of the device 10 will be explained by an operation example including some experimental figures and numbers. These numbers are given only as an example, and the skilled person will understand that other numbers may be used.

If hemofiltration is performed during 4 hours (240 min), an ultrafiltration volume of about 1600 ml may be appropriate, corresponding to 6.7 ml/min.

In order to obtain a desired dose of dialysis, a postinfusion of about 8000 ml may be appropriate, corresponding to 33.3 ml/min during 4 hours.

The sterile fluid in fluid bag 12 is produced on-line by means of the device 10 according to an embodiment of the invention, as described below.

Initially, fluid bag 12 comprises about 2.2 liter conventional hemofiltration fluid, comprising for example ions of sodium (140 mM), potassium (4 mM), calcium (2.75 mM), magnesium (1.5 mM), chloride (108 mM), and bicarbonate (32 mM) and possibly glucose. Any other recipe can be prescribed.

Hemofiltration fluid is infused at a predetermined rate of for example 33.3 ml/min. Simultaneously, fluid is removed from the hemofilter as a filtrate via outlet 9 by means of pump 11, for example at a rate of 40 ml/min, resulting in an ultrafiltration of 6.7 ml/min.

The filtrate fluid is accumulated in a filtrate bag 14, until there is 1.4 liters of filtrate fluid in bag 14, which takes about 30 minutes. The filtrate bag 14 initially comprises 0.2 liter.

The filtrate bag 14 is connected to an evaporator bag 31 via a line 15 provided with a valve 16. The evaporator bag 31 is arranged in an evaporator/condenser device 30 to be described in further details below. The evaporator bag 31 is arranged at a lower position compared with the filtrate bag 14 so that when valve 16 is opened, fluid passes from filtrate bag 14 to evaporation bag 31 by gravity, until 1.2 liter has passed to evaporator bag 31, whereupon valve 16 is closed (which may take about 2 min). Filtrate pump 11 continues to pump filtrate fluid to filtrate bag 14 all the time.

When valve 16 is closed, a pump 32 lowers the pressure in an enclosure 33 surrounding the evaporator bag 31 (which may take about 0.5 min). The enclosure comprises a medium, such as oil, which is pumped from the enclosure 33 to a sump 35 in order to lower the pressure to an absolute pressure of about 50 mmHg.

Heat energy is provided by a heat source 34 for evaporation or boiling of the contents of the evaporator bag 31. It takes about 2260 kJ to boil 1 kg of water (1 liter), which may be provided by a heat energy source of 1507 W during 25 minutes (neglecting losses). At a pressure of 50 mmHg, water boils at about 38° C., which means that the oil surrounding the evaporator bag 31 may have a temperature of about 45° C. in order to provide a required temperature gradient.

Evaporator bag 31 is connected with a condenser bag 36 via a steam line 37 and steam may pass from evaporator bag 31 to condenser bag 36 driven by a pressure difference. The condenser bag 36 is maintained at (substantially) the same pressure as the evaporator bag 31 and a cooling device 38 is arranged to cool the condenser bag 36 for condensation of steam entering the condenser bag 36 via steam line 37. The same amount of energy, 2260 kJ needs to be removed, which means that the oil surrounding the condenser bag 36 may have a temperature of about 31° C. in order to provide the required temperature gradient.

When 1000 ml of water has boiled and been transferred from the evaporator bag 31 to the condenser bag 36, which takes about 25 minutes the process is interrupted and the enclosure 33 is returned to atmospheric pressure by operating the oil pump 32 (which takes about 0.5 min). Now, there is 200 ml of filtrate fluid left in the evaporator bag 31 and 1000 ml of pure water in the condenser bag 36.

Then, the condenser bag 36 is connected to the fluid bag 12 via a line 17 provided with a valve 18. When valve 18 is opened, the water in the condenser bag 36 flows by gravity to the fluid bag 12, whereby 1000 ml of water is transferred from condenser bag 36 to fluid bag 12.

At the same time a concentrate pump 19 is operated for providing 33 ml of a concentrate fluid included in a concentrate bag 20 to the fluid bag 12 via a line 21. The concentrate fluid has a composition so that the composition of substances (ions) mentioned above for a replacement fluid for hemofiltration is obtained when the concentrate fluid is mixed with pure water from the condenser bag. Thus, the concentrations of substances (ions) in said concentrate fluid may be 33 times the mentioned concentrations.

The fluid in the condenser bag 36 is essentially pure water without any ions or substances dissolved therein. It may happen that some water droplets follows the steam in the steam line 37 and contaminate the pure water, but such substances and ions are not detrimental for the operation. It is noted that the pure water has at least 95% purity or 99% purity.

Simultaneously, the contents of the evaporator bag 31 is drained to a drain bag 22 via a line provided with a valve 24. About 200 ml is drained to the drain bag (which may take 2 min).

After 30 minutes, the process is repeated—until the treatment is finalized after 240 min (8 cycles).

In the meantime, pumps 13 and 11 are operating all the time, whereupon the fluid level in fluid bag 12 decreases and the fluid level in filtrate bag 14 increases.

Since the evaporation/condensation takes about 25 minutes, there is 5 minutes for transferring the fluids to the evaporator bag and for transferring the fluids from the condenser bag to the fluid bag 12 and from the evaporator bag to the drain bag.

After 8 cycles, the contents of the fluid bag 12 has increased by 8*33 ml=264 ml and the end volume in fluid bag 12 is 1464 ml of fluid (inclusive of 200 ml from the start fluid).

The filtrate bag 14 comprises 200 ml of filtrate, which is discarded.

The drain bag 22 comprises 1600 ml of a fluid which mimics or is proportional to the plasma in the blood and has passed the pores of the hemofilter. In particular, the concentration of urea, sodium, potassium, calcium and magnesium is six times the concentration in blood.

The process is controlled by a processor, not shown, which receives input signals from level detectors, sensing the fluid levels in evaporator bag 31 and condenser bag 36 and drain bag 22. In addition, level detectors may be arranged at filtrate bag 14 and fluid bag 12 in order to ensure that these bags always comprises a minimum amount of fluid (for example 200 ml as indicated above). Furthermore, temperature sensors may be arranged to measure the temperature at the evaporator bag and condenser bag to ensure correct operation.

Thus, a hemofiltration process is provided in which the infusate or replacement fluid is produced (intermittently) on-line by the evaporation/condensation device and by which the ultrafiltration fluid is concentrated at the same time.

In an embodiment, about 250 mmole of urea should be removed per day. If the concentration of urea in blood, and consequently the concentration of urea in the filtrate is about 26 mM (millimole per liter), there is removed:

26 mmole/1*6 (concentration)*1,6 liter=249.6 mmole

It is believed that the process according to the embodiment of FIG. 1 works best in postdilution, but also predilution may be used (not shown).

The pure water in condenser bag 17 has a temperature of about 38° C., which is convenient for the hemofiltration process. The pressure during the process determines the temperature.

However, the evaporator/condensor device 10 may be operated at a higher pressure, for example 70 mmHg corresponding to a temperature of about 44° C., since the pure water is cooled when being mixed with the remaining fluid in the fluid bag 12 and with the concentrate fluid from the concentrate bag 20. Cooling of the condenser may be facilitated at higher temperature.

The evaporator/condensor device 10 may be operated at a still higher pressure, up to atmospheric pressure. In this embodiment, a cooling of the pure water exiting the condensor bag may be required. A heat pump may be used to recover the heat energy and use the recovered heat energy for heating of the evaporator bag. This also applies to all embodiments described below.

Alternatively, the evaporator/condensor device may be operated at a lower pressure, for example 30 mmHg.

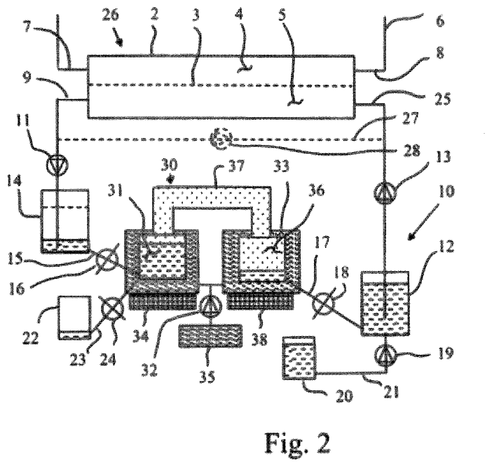

The embodiment according to FIG. 1 may be modified to operate in a dialysis mode or diafiltration mode, as shown in FIG. 2. The same components as in the embodiment of FIG. 1 has the same reference numerals. The outlet flow from pump 13 is diverted to a dialysate inlet 25 of a dialyzer 26. A recirculation line 27 operated by a pump 28 may be arranged as shown in broken lines. In other respects, the operation is substantially the same. However, different flow rates and times may be used.

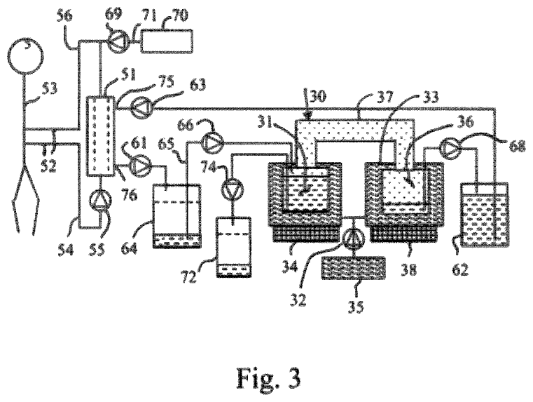

A further configuration is shown in FIG. 3 wherein the device is operating in a peritoneal dialysis circuit.

The evaporator/condenser device 30 is the same as in the embodiments of FIGS. 1 and 2 and its components have the same reference numerals. However, the valves 16, 18, 24 are replace by pumps 66, 68 and 74, which, however, performs the same function as the valves 16, 18, 24 in the previous embodiments. However, the fluid flows are no longer dependent on gravity.

The device 60 according to the embodiment of FIG. 3 is connected to a double lumen catheter 52 arranged in a patient 53 for connection with a peritoneal fluid arranged in a peritoneal cavity of the patient. The peritoneal fluid passes via a line 54 comprising a circulation pump 55 to a dialyzer 51 and back to the patient via a line 56. The circulation pump 55 is arranged to provide a circulation flow rate, which is sufficient for exchange of substances over the dialyzer. Such a flow rate may be for example about 50 ml/min, which is well tolerated by the patient.

A concentrate pump 69 is connected to a concentrate bag 70 via line 71. Concentrate pump 69 is arranged to infuse a concentrate fluid (for example with similar composition and concentration as bag 20) into the fluid line 56 for circulating conditioned peritoneal fluid back to the patient. The concentrate fluid has a composition calculated for replenishing required substances, such as glucose, sodium, etc. Examples of suitable substances is disclosed in patent publication WO2016190794A2. Other recipes may be used.

A dialysate pump 61 is connected to a dialysate outlet 76 of the dialyzer 51 for removing dialysate fluid from the dialyzer 51 at a predetermined rate of for example 19 ml/min. The pump 61 delivers the dialysate into a dialysate bag 64 until 570 ml has been provided during 30 min.

Simultaneously, a return pump 63 connected to a fluid bag 62 provides pure water to a dialysate inlet 75 of the dialyzer, for example 16.7 ml/min.

The evaporator/condenser device 60 according to FIG. 3 may be operated with the following data: 570 ml of filtrate fluid is provided each 30 minutes by pump 66. The heat source 34 provides a power of 754 W during 25 minute and cooling device 38 removes 754 W in order to provide 500 ml of pure water in condenser bag 36 and retain 70 ml of ultrafiltration fluid in evaporator bag, which is concentrated 8 times. The ultrafiltration fluid (70 ml each 30 min) is given off to a drain bag 72 by means of a drain pump 74 and pure water in condenser bag 36 is given off by pump 68 to fluid bag 62 (500 ml each 30 min).

Thus, 140 ml of drain fluid and 1000 ml of pure water are provided each hour. The ultrafiltration fluid is concentrated eight times. The concentrate fluid is infused with a rate of 0.66 ml/min (40 ml/h). Thus, the net ultrafiltration will be 100 ml/h. If the device is operated during 10 hours per day, an ultrafiltration of 1,0 liter is obtained.

As mentioned above, the evaporator/condenser device 60 is operated intermittently in cycles of 30 min. However, the cycle may be larger, such as 60 min, or smaller such as 15 or 20 min. A larger cycle results in that more pure fluid is required from the start in the fluid bag 62.

In this embodiment (and the following embodiments), it is not required to return the evaporator/condenser bags to ambient pressure, but the evaporator/condenser can be maintained at a low pressure during filling and draining of the evaporator bag and the emptying of the condenser bag, because the pumps may be operated to counteract such pressure differences. Any increase of the pressure during such pump steps may be taken care of as indicated below.

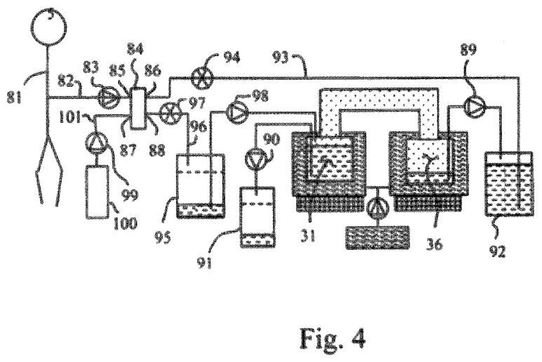

A further embodiment is shown in FIG. 4 for providing dialyzing capacity to a peritoneal ultrafiltration device as shown in patent publications WO2015130205A1 and WO2016080883A1. A peritoneal ultrafiltration device according to said patent publications comprises a device for (intermittently) infusing glucose into peritoneal fluid for maintaining an osmotic gradient substantially constant in the peritoneal cavity. Peritoneal fluid is periodically withdrawn from the peritoneal cavity whereupon concentrated glucose is metered to the withdrawn peritoneal fluid before or during reinfusion to the peritoneal cavity. In this manner, the glucose concentration in the peritoneal cavity can be maintained substantially constant.

The embodiment shown in FIG. 4 comprises a fluid line 82 connecting to a peritoneal catheter of a patient 81. The catheter may be single lumen as shown or double lumen.

The fluid line comprises a fluid pump 83 connecting to a first inlet/outlet 85 of a manifold 84. A second inlet/outlet 86 is connected via a line 93 to a water bag 92. Line 93 comprises a valve 94. A third inlet/outlet 87 of manifold 84 is connected to a concentrate bag 100 via a line 101 provided with a concentrate pump 99. A fourth inlet/outlet 88 of manifold 84 is connected to a drain bag 95 via a line 96 and a valve 97.

The drain bag 95 is via a drain pump 98 connected to an evaporator bag 31 of a evaporator/condenser device of the same construction as in previous embodiments. An condenser bag 36 is connected to the water bag 92 via a pure water pump 89. The evaporator bag 31 is further connected to an ultrafiltration bag 91 via an ultrafiltration pump 90.

The device according to FIG. 4 operates as follows:

In a first phase, concentrate pump 99 is non-operated while fluid valve 94 is closed and drain valve 97 is open. Fluid pump 83 is operated in its forward direction, whereby 285 ml of peritoneal fluid is pumped from the peritoneal cavity into drain bag 95. Simultaneously, ultrafiltration pump 90 is operated to drain a residue maintained at the bottom of evaporator bag, which is 35 ml of ultrafiltration fluid left from a previous cycle. Such 35 ml of ultrafiltration fluid is pumped to ultrafiltration bag 91 by pump 90, whereupon the evaporator bag becomes empty. Still simultaneously, pure water pump 89 pumps 250 ml of pure water (from a previous cycle) from condenser bag 36 to water bag 92.

In a second phase, drain valve 97 is closed and water valve 94 is opened. Fluid pump 83 is operated in a reverse direction to pump 250 ml of pure water from water bag 92 into the peritoneal cavity of the patient via line 82 and the catheter. Simultaneously, concentrate pump 99 is operated in order to replenish the pure water with 10 ml of glucose and other ions (sodium, potassium, calcium, magnesium, acetate, glucose, etc) comprised in the concentrate bag 100. Simultaneously, drain pump 98 pumps 285 ml of fluid from drain bag 95 into the evaporator bag 31.

In a third phase, all pumps are stopped and all valves are closed, whereupon the evaporation/condenser device is operated to boil water in the evaporator bag and condense steam in the condenser bag, until 250 ml of pure water has been condensed in condenser bag, which takes about 10 minutes and requires 942 W of heating/cooling. The first and second phase each takes 2.5 min. Thus, a total cycle of 15 minutes is obtained. The peritoneal fluid inside the peritoneal cavity is left to equilibrate during 10 minutes in the third phase.

Finally, the three phases are repeated a desired number of times.

The process may be altered by interleaving a cycle in which only drain and fill takes place and the evaporator/condenser device is operated only each second cycle, with double amounts.

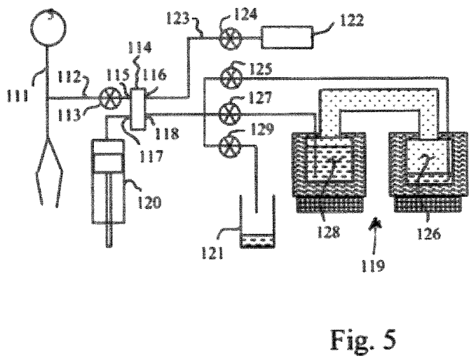

FIG. 5 shows an embodiment similar to the embodiment according to FIG. 4 but in which a single pump performs all actions.

The embodiment shown in FIG. 5 comprises a fluid line 112 connecting to a peritoneal catheter of a patient 111. The catheter may be single lumen as shown or double lumen. The fluid line comprises a fluid valve 113 connected to a first inlet/outlet 115 of a manifold 114. A second inlet/outlet 116 is connected via a line 123 to a concentrate bag 122. Line 123 comprises a concentrate valve 124. A third inlet/outlet 117 of manifold 114 is connected to a piston pump 120 (for example a manually operated syringe). A fourth inlet/outlet 118 of manifold 114 is connected to three valves, a condenser valve 125 connecting to a condenser bag 126 of the evaporator/condenser device 119, an evaporator valve 127 connecting to an evaporator bag 128 of the evaporator/condenser device 119, and a drain valve 129 connecting to a drain bag 121, which is open to the atmosphere.

The device is operated as follows:

During all steps only one valve is opened while the other valves are closed. Thus, only the open valve is indicated below.

In a first phase, evaporator valve 127 is opened and the piston 120 is withdrawn to empty any residual fluid in the evaporator bag 128, which normally is 35 ml of ultrafiltration peritoneal fluid from a previous cycle.

In a second phase, the drain valve 129 is opened and the contents of the piston 120 (35 ml) is transferred to the drain bag 121.

In a third phase, the fluid valve 113 is opened and piston 120 is operated to withdraw 285 ml of fluid from the peritoneal cavity.

In a fourth phase, evaporator valve 127 is opened and the contents (285 ml) of the piston 120 is transferred to the evaporator bag 128, which was emptied in the first phase.

In a fifth phase, concentrate valve 124 is opened and the piston is operated to withdraw a small amount (10 ml) of concentrate fluid into the piston 120.

In a sixth phase, condenser valve 125 is opened and the piston is operated to withdraw all (250 ml) of pure water from the condenser bag 126, which mixes with the concentrate fluid to provide a diluted fluid suitable to be introduced into the peritoneal cavity to (partly) replenish the fluid therein.

In a seventh phase, fluid valve 113 is opened and the piston is operated to fill the contents (260 ml) of the piston into the peritoneal cavity.

In an eight phase, condenser valve 125 is opened and the piston 120 is retracted in order to remove steam from condenser bag 126 in order to reduce the pressure therein to 50 mmHg. If this pressure reduction step requires several steps of piston retraction, the piston may be emptied in the drain bag 121 by opening drain valve 129, since drain bag is open to the atmosphere.

In a ninth phase, the evaporator/condenser device is operated by providing 942 W heat energy at a temperature of about 45° C. and removing 942 W at a temperature of about 31° C., thereby boiling the contents of evaporator bag and condensing steam in the condenser bag until 250 ml of water has been transferred to condenser bag.

The phases are repeated as many times as desired with a minimum cycle time of about 15 min, required for operating the evaporator/condenser device.

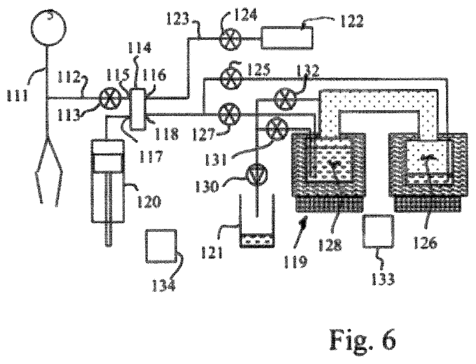

A further modified embodiment is shown in FIG. 6. A second pump 130 is arranged to remove the ultrafiltration peritoneal fluid from evaporator bag 128 instead of the first and second phase mentioned above. Thus, a drain valve 131 connects the second pump 130 to the bottom of evaporator bag 128.

A pressure valve 132 is arranged to connect the pump 130 to the upper portion of evaporator bag 128 when the pressure therein should be lowered to 50 mmHg. The second pump 130 may be a peristaltic pump or another pump capable of providing a subpressure of absolute 50 mmHg also when operating on a gas, such as steam, for example a centrifugal pump or membrane pump.

As mentioned above, the condenser bag is cooled to provide condensed steam, namely pure water. The cooling energy may be absorbed by a heat pump 133 with its cold side arranged at the condenser bag 126 and its warm side arranged at the evaporator bag 128. The heat pump is arranged to absorb heat energy at a temperature of about 31° C. and give of the heat energy at a temperature of about 45° C. Such a heat pump may have a high heat factor of more than 5. In this manner, the energy consumption required for the process may be reduced.

A conventional compressor-driven heat pump may be used. Alternatively, the heat pump may be arranged in the nature of a Peltier element.

The process is driven by a computer 134 schematically shown in FIG. 6.

It is noted, that the evaporation process may required larger amount of energy for evaporating a fluid comprising substantial amounts of ions, in particular sodium ions. Thus, the supply of power is increased for compensating for such effect.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and other embodiments than those specified above are equally possible within the scope of these appended claims.

1. A dialysis device comprising a dialysis circuit for providing dialysis fluid and returning purified fluid to the dialysis circuit, and an evaporator/condenser device, which comprises:
an evaporator bag for receiving dialysis fluid from the dialysis circuit and for evaporation thereof for producing steam; and
a condenser bag for receiving said steam produced in the evaporation bag and condensing the steam for producing pure water in said condenser bag for returning to said dialysis circuit;
further comprising
a drain bag for receiving a concentrated ultrafiltration fluid produced in the evaporator bag.

2. The dialysis device according to claim 1, wherein
said evaporation and said condensing takes place at a subatmospheric pressure, between 30 and 70 mmHg.

3. The dialysis device according to claim 1, wherein said pure water is conditioned by a concentrate fluid before being returned to the dialysis circuit, wherein said concentrate fluid comprises at least one of: sodium ions, potassium ions, calcium ions, magnesium ions, chloride ions, bicarbonate ions, acetate, glucose.

4. The dialysis device according to claim 1, wherein said evaporator/condenser device is operated batchwise, so that a predetermined amount of dialysis fluid is evaporated and condensed in a single step.

5. The dialysis device according to claim 1, wherein said dialysis circuit is a hemofiltration dialysis circuit comprising a hemofilter providing said dialysis fluid and wherein said pure water, conditioned with concentrate fluid, is provided as postdilution or predilution.

6. The dialysis device according to claim 1, wherein said dialysis circuit is a peritoneal dialysis circuit.

7. The dialysis device according to claim 1, wherein said ultrafiltration dialysis fluid, which is drained to a drain bag, is concentrated at least five times, such as eight times or ten times.

8. The dialysis device according to claim 1, wherein said evaporation and said condensation takes place at substantially the same pressure.
